(19) United States
(12) Patent Application Publication
(10) Pub. No.: US 2023/0242475 A1
(43) Pub. Date: Aug. 3, 2023

(54) Compositions Containing Dialkyl Amino Acid Ester Salts

(71) Applicant: Stepan Company, Northbrook, IL (US)

(72) Inventors: Dennis S. Murphy, Libertyville, IL (US); Renata A. Butikas, Chicago, IL (US); Patrick Shane Wolfe, Palatine, IL (US); Matthew V. Vollmer, Morton Grove, IL (US)

(21) Appl. No.: 18/120,697

(22) Filed: Mar. 13, 2023

Related U.S. Application Data
(63) Continuation of application No. PCT/US2021/050042, filed on Sep. 13, 2021.
(60) Provisional application No. 63/077,809, filed on Sep. 14, 2020.

(51) Int. Cl. C07C 229/24; C07C 227/18; A61K 8/44; A61Q 5/12

(52) U.S. Cl. CPC C07C 229/24; A61K 8/44; A61Q 5/12; C07C 227/18

(57) ABSTRACT

Compositions comprising at least one dialkyl amino acid ester salt as a cationic active are disclosed. The compositions are useful for hair care, as well as in other applications, such as cleaning compositions, fabric softening compositions, and skin care compositions. The dialkyl amino acid ester salts are derived from the esterification reaction of an amino acid having at least two carboxylic acid groups with a fatty alcohol, wherein the amine group of the amino acid is protonated with an acid. The compositions may further include a glyceride component comprising monoglycerides, diglycerides, or a combination thereof.

### RELATED APPLICATIONS

This application is a continuation of and claims priority to PCT Application No. PCT/US21/50042 filed on Sep. 13, 2021, which claims priority to U.S. Provisional Application No. 63/077,809, filed Sep. 14, 2020, both of which are incorporated herein by reference in their entirety.

### FIELD OF THE INVENTION

The present technology relates to dialkyl amino acid ester salts that are useful for providing conditioning, softening, and/or cleaning properties in compositions. In particular, the present technology relates to dialkyl amino acid ester salts that are the neutralized (protonated) reaction product of an amino acid having at least two carboxylic acid groups and a fatty alcohol. The dialkyl amino acid ester salts can be used for hair care, as well as other applications, such as cleaning compositions, fabric softening compositions, and skin care compositions.

### BACKGROUND OF THE INVENTION

There has been a trend in the personal care industry to formulate compositions with ingredients that are based on renewable resources derived from plants or animals, rather than fossil fuels. Such ingredients are considered “green” or “natural”, since they are derived from renewable and/or sustainable sources. As a result, they are more environmentally friendly than ingredients derived from fossil fuels, particularly if they are also manufactured without the need for petroleum-derived solvents. An ingredient having a high Biorenewable Carbon Index (BCI), such as greater than 80, indicates that the ingredient contains carbons that are derived primarily from plant, animal or marine-based sources.

An example of a natural ingredient derived from renewable sources is a neutralized amino acid ester that is obtained from the reaction product of a neutral amino acid having a non-polar side chain reacted with a long chain fatty alcohol. U.S. Pat. No. 8,105,569 describes such neutralized amino acid esters. The neutralized amino acid esters are cationic, and therefore could potentially replace traditional cationic hair conditioning agents, such as behentrimonium chloride (BTAC) and cetrimonium chloride (CETAC), which traditionally have unfavorable environmental profiles.

One drawback of the neutralized amino acid ester is that it is a more expensive cationic ingredient than other cationic components typically used in hair care compositions, such as quaternary ammonium compounds and amidoamines. In addition, a greater amount of the neutralized amino acid ester, compared to the traditional cationic active agents, is often required to achieve acceptable performance. Using more of an ingredient that is already more costly results in a more expensive product to manufacture. It would therefore be desirable to have composition actives that are derived from renewable sources, but that can also deliver acceptable performance at lower cost, and at use levels comparable to the use levels used for traditional cationic active agents.

### SUMMARY OF THE INVENTION

The present technology is directed to dialkyl amino acid ester salts that are the reaction product of a neutralized (protonated) amino acid and a fatty alcohol. The dialkyl amino acid ester salts can be used in compositions as a cationic component either alone or in combination with a glyceride component. The glyceride component comprises monoglycerides, diglycerides, or mixtures thereof, and optionally, from 0 to 50% by weight triglycerides, based on the total weight of the glycerides. In some embodiments, combining glycerides with the dialkyl amino acid ester salt can improve the wet combing properties of the dialkyl amino acid ester salt.

In one aspect, the present technology is directed to a composition comprising:
(a) 0.01% to about 50% by weight of a cationic active component comprising a dialkyl amino acid ester salt having the following chemical formula:

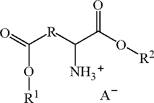

wherein R is a linear or branched carbon chain containing 1 to 10 carbon atoms, R¹ and R² are independently C8 to C22 linear or branched alkyl groups, and A⁻ is the anion of a proton-donating acid;
(b) optionally, one or more additional components; and
(c) diluent to balance the composition to 100%. In one embodiment, the composition is a hair conditioning composition.

In a further aspect, the present technology is directed to a method of making a dialkyl amino acid ester salt comprising the steps of:
(a) providing an amino acid having at least two carboxylic acid groups;
(b) providing a fatty alcohol feedstock, wherein the fatty alcohol feedstock comprises one or more linear or branched, saturated or unsaturated fatty alcohols having from 8 to about 22 carbon atoms;
(c) providing a proton-donating acid to protonate the amine group of the amino acid; and
(d) in the absence of added solvent, reacting the protonated amino acid with the fatty alcohol feedstock to form the dialkyl amino acid ester salt.

A further aspect of the present technology is a dialkyl amino acid ester salt having the following chemical formula:

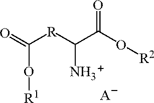

wherein R is a carbon chain containing 1 or 2 carbon atoms, R1 and R2 are independently C8 to C22, preferably C8 to C16, linear or branched alkyl groups, and A- is ethane sulfonate or methane sulfonate.

### DETAILED DESCRIPTION OF THE PREFERRED EMBODIMENTS

The compositions of the present technology comprise one or more dialkyl amino acid ester salts that are derived from biorenewable sources, and provide conditioning, softening, or cleaning performance.

“Biorenewable Carbon Index” (BCI) refers to a calculation of the percent carbon derived from a biorenewable resource, and is calculated based on the number of biorenewable carbons divided by the total number of carbons in the entire molecule.

“Biorenewable” is defined herein as originating from animal, plant, or marine material.

The dialkyl amino acid ester salts of the present technology are obtained by esterification of (i) an amino acid having at least two carboxylic acid groups with (ii) a fatty alcohol, wherein the amine group of the amino acid has been protonated with an acid. The dialkyl amino acid ester salts of the present technology may be represented by the structure of Formula (1):

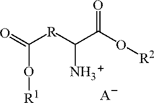

wherein R is a linear or branched carbon chain containing 1 to 10 carbon atoms, R¹ and R² are independently linear or branched alkyl groups containing 8 to 22 carbon atoms, preferably 8 to 16 carbon atoms, most preferably 8 to 14 carbon atoms, and A⁻ is the anion of a proton-donating acid, preferably ethanesulfonate. The dialkyl amino acid ester salts of Formula 1 can have a selected distribution of the R¹ and R² alkyl groups. For example, in a given sample, at least 50 mol% of the R¹ and R² alkyl groups have from 8 to 16 carbon atoms, based on the total number of moles of R¹ and R² alkyl groups in the sample. Alternatively, at least 60 mol%, alternatively at least 70 mol%, alternatively at least 80 mol%, alternatively at least 90 mol%, alternatively at least 95 mol% of the total number of moles of the R¹ and R² alkyl groups have from 8 to 16 carbon atoms. In some embodiments, 100% of the total number of moles of the R¹ and R² alkyl groups in a given sample have from 8 to 16 carbon atoms (i.e., the R¹ and R² alkyl groups contain no C18-C22 alkyl groups). In other embodiments, 100% of the total number of moles of the R¹ and R² alkyl groups have from 8 to 14 carbon atoms. In some embodiments, 100% of the total numbers of moles of the R¹ and R² alkyl groups have from 12 to 16 carbon atoms The dialkyl amino acid ester salts of Formula 1 can also have a selected distribution of the R¹ and R² alkyl groups wherein at least 80 mol% of the dialkyl amino acid ester salt molecules have from 24 to 30 carbon atoms in the combined R¹ and R² alkyl groups. The carbon chain distribution of the R¹ and R² alkyl groups is based upon the carbon chain distribution of the starting fatty alcohol reactant, which can be determined by gas chromatography.

Preferably, the R¹ and R² alkyl groups are derived from a fatty acid source having an iodine value of less than 3. The iodine value represents the mean iodine value of the fatty acid source for the fatty alcohol feedstock. Most preferably, the R¹ and R² groups are fully hydrogenated. “Fully hydrogenated” means that any double bonds present have been almost completely removed by hydrogenation, but does not preclude the possibility that a small percentage of double bonds may remain. Although less preferred, the R¹ and R² alkyl groups may be derived from a fatty acid source having an iodine value of greater than 3, i.e. the fatty acid source for the fatty alcohol feedstock has at least some double bonds, provided the esterification reaction between the fatty alcohol and the amino acid is performed under non-acidic conditions.

Esterification can optionally be facilitated by the use of catalysts including, but not limited to, titanium-based catalysts, such as those sold by E.I. DuPont de Nemours and Company under the name TYZOR®, for example, titanium t-butoxide (TYZOR®) or ammonium salt of lactic acid chelate of titanium dihydroxide (TYZOR® LA), and tin-based catalysts, such as dioctyltin bis-(2-ethylhexanoate) or dioctyltin dilaurate, available from REAXIS Inc., McDonald, PA.

Amino acids for the formation of the ester can be any that have at least two carboxylic acid groups. Particular amino acids include L-aspartic acid and L-glutamic acid.

To obtain the dialkyl amino acid ester salt, the amine group of the amino acid is preferably neutralized with an acid, and the neutralized amino acid is reacted with one or more fatty alcohols. Suitable fatty alcohols may be linear or branched, and may additionally be saturated and/or unsaturated, preferably saturated. The fatty alcohol can contain about 8 to about 22 carbon atoms, preferably 8 to 16 carbon atoms. Specific examples of fatty alcohols that can be used include caprylic alcohol, capric alcohol, lauryl alcohol, myristyl alcohol, palmityl alcohol, stearyl alcohol, brassica alcohol, or mixtures or combinations thereof. Preferably, the fatty alcohols are derived from non-petrochemical sources. In some embodiments, the fatty alcohol is a mixture of fatty alcohols wherein between 65 wt% and 75 wt% of the alkyl groups in the fatty alcohol have 12 carbon atoms, between 20 wt% and 30 wt% of the alkyl groups have 14 carbon atoms, and between 3 wt% and 8 wt% of the alkyl groups have 16 carbon atoms, based on the total weight of alkyl groups in the fatty alcohol mixture. In some embodiments, the fatty alcohol can be derived from a coconut source, comprising a mixture of fatty acids having carbon chain lengths of 8 to 18 carbon atoms. The molar ratio of fatty alcohol reacted with amino acid is about 1.6:1 to about 4.5:1, alternatively about 1.7:1 to about 3.5:1, alternatively about 1.8:1 to about 3:1.

The amine group of the dialkyl amino acid ester may be fully or partially neutralized by an acid, to facilitate its cationic behavior. Any acid may be used, including organic and inorganic acids. Examples of acids include, but are not limited to, lactic acid, citric acid, maleic acid, adipic acid, boric acid, glycolic acid, formic acid, acetic acid, ascorbic acid, uric acid, oxalic acid, butyric acid, oxalic acid, formic acid, methane sulfonic acid, ethane sulfonic acid, higher alkyl analogs of ethane sulfonic acid, such as, but not limited to propane sulfonic acid, butane sulfonic acid, hydrochloric acid, sulfuric acid, nitric acid, phosphoric acid, or combinations thereof. In some embodiments, the acid is ethane sulfonic acid.

In some embodiments, the neutralized dialkyl amino acid ester salt is dialkylaspartate ethanyl sulfonate or dialkylglutamate ethanyl sulfonate, wherein the alkyl groups bound to the amino acid have a combined carbon chain distribution comprising between 65 wt% and 75 wt% C12, between 20 wt% and 30 wt% C14, and between 3 wt% and 8 wt% C16, based on the total weight of alkyl groups. Some preferred neutralized dialkyl amino acid esters include dilaurylaspartate ethanyl sulfonate, or dilaurylglutamate ethanyl sulfonate. Dilauryl aspartate ethanyl sulfonate can be prepared from the esterification of lauryl alcohol with L-aspartate ethanyl sulfonate. L-aspartate ethanyl sulfonate may be prepared by reacting the amine group on aspartic acid with ethanesulfonic acid. No solvent is necessary for the preparation of dialkylaspartate ethanyl sulfonate.

In some embodiments of the present technology, the dialkyl amino acid ester salts can be combined with a glyceride component. The glyceride component may comprise monoglycerides, diglycerides, or mixtures thereof. Optionally, triglycerides may also be included in the glyceride component. An amount of triglycerides in the glyceride component can range from 0 to about 50% by weight, alternatively 0 to about 40% by weight, alternatively 0 to about 30% by weight, alternatively 1% to about 50% by weight, alternatively about 1% to about 40%, about 1% to about 30%, about 1% to about 20%, or about 1% to about 10% by weight, based on the total weight of the glyceride component. The monoglycerides, diglycerides, or triglycerides, or combinations thereof, comprise saturated, unsaturated, or a mixture of unsaturated and saturated fatty acid carboxylate groups containing about 8 to about 32 carbon atoms. In some embodiments, the fatty acid groups comprise at least 50% by weight, alternatively at least 60% by weight, unsaturated fatty acid groups having at least one carbon-carbon double bond. In some embodiments, the fatty acid groups are derived from oleic acid. In some embodiments, the glyceride component is a mixture of monoglycerides and diglycerides. The ratio of monoglyceride to diglyceride in the mixture can be about 1:3 to 3:1, although in some embodiments, a ratio of about 1:1 monoglyceride to diglyceride is preferred. When the dialkyl amino acid ester salt is combined with a glyceride component, the mixture comprises about 50% to 95%, alternatively about 50% to about 90%, alternatively about 55% to about 90%, alternatively about 60% to about 90% by weight of dialkyl amino acid ester salt, and about 5% to about 50%, alternatively about 10% to about 50%, alternatively about 10% to about 45%, alternatively about 10% to about 40% by weight of the glyceride component, based on the combined weight of the dialkyl amino acid ester salt and glyceride component.

The dialkyl amino acid ester salt can be used as is, as an active component, or diluted in particular solvents. In some embodiments, the solvents are those suitable for personal care. Examples of solvents for diluting the dialkyl amino acid ester salt include, but are not limited to, propylene glycol, 1,3-propandiol, glycol ethers, glycerin, sorbitan esters, lactic acid, alkyl lactyl lactates, isopropyl alcohol, ethyl alcohol, dimethyl adipate, oleyl alcohol, 1,2-isopropylidine glycerol, benzyl alcohol, dimethyl lauramide myristamide, N-butyl lactate, citrate esters, dimethyl lactide, laureth-2 lactide, 1,2-butylene carbonate, conjugated linoleic acid, isosorbide dimethyl ether, propylene carbonate, C6-C18 methyl esters, C12-15 alkyl benzoate, glycerol monooleate, triglyceride oils, including, but not limited to, sunflower oil, borage oil, moringa oil, argan oil, or raddish seed oil, jojoba oil, sunflower oil/MDEA esteramine, or combinations thereof.

When used, the amount of solvent can range from about 1% to about 70%, alternatively about 5% to about 70%, alternatively about 10% to about 60%, alternatively about 10% to about 50%, alternatively about 10% to about 40%, alternatively about 10% to about 30% by weight, and the amount of the dialkyl amino acid ester salt can range from about 30% to 99%, alternatively about 30% to about 95%, alternatively about 40% to about 90%, alternatively about 50% to about 90%, alternatively about 60% to about 90%, alternatively about 70% to about 90% by weight, based on the combined weight of the dialkyl amino acid ester salt and solvent. In some embodiments, the amount of solvent is about 1% to about 50% by weight, and the amount of the dialkyl amino acid ester salt is about 50% to about 99% by weight.

The dialkyl amino acid ester salts of the present technology can be formulated into hair care compositions including, but not limited to, hair conditioners and hair repair compositions. The dialkyl amino acid ester salts could also be formulated into other end use products such as, but not limited to, fabric softeners, fabric conditioners, hard surface cleaners, and skin care compositions. It is expected that the dialkyl amino acid salts will also work well as cationic emulsifiers. Being that dialkyl amino acid salts will act as deposition aids to surfaces, they could be used to enhance or more efficiently utilize the active ingredients for: SPF in sun screens, skin moisturization for lotions, color benefits from pigments used in cosmetics, anti-itch in topical treatments such as Benadryl® products available from Johnson & Johnson Consumer Inc., insect repellency from topically applied products such as OFF® available from S.C. Johnson & Son, Inc., wound healing from topically applied anti-bacterial/anti-fungal treatments, hand sanitization for products which rely on cationic biocidal actives, and the like. Product compositions can include the dialkyl amino acid ester salt in an amount of about 0.01% to about 50% by weight of the product composition, alternatively about 0.05% to about 25%, alternatively about 0.1% to about 12%, alternatively about 0.01% to about 10%, alternatively about 0.1% to about 5%, alternatively about 0.5% to about 5%, alternatively about 1% to about 5%, alternatively about 1% to about 4% by weight of the composition. When glycerides are included in the product composition, the combination of dialkyl amino acid ester salt and glycerides can comprise about 0.01% to about 17% by weight of the composition, alternatively about 0.01% to about 12%, alternatively about 0.1% to about 7%, alternatively about 0.7% to about 5% by weight of the composition.

The compositions may contain other optional ingredients suitable for use, such as surfactants or other additives, and a diluent, such as water. Examples of surfactants include nonionic, cationic, anionic, and amphoteric surfactants, or combinations thereof. If anionic surfactants are included in the composition, the ratio of cationic salt to anionic surfactant in the composition is preferably at least 2:1. Examples of nonionic surfactants include, but are not limited to, fatty alcohol alkoxylates, polyalkylene glycols, mono- and/or dialkyl sulfosuccinates, fatty acid isethionates, fatty acid sarcosinates, fatty acid glutamates, ether carboxylic acids, alkyl oligoglucosides, and combinations thereof. Examples of cationics include, but are not limited to, BTAC, CETAC, and polyquaterniums, or combinations thereof. Examples of anionic surfactants include, but are not limited to, alkyl sulfates, alkyl ether sulfates, alpha sulfonated fatty acid esters, sulfonated alpha olefins, acyl methyl taurates, acyl isethionates, acyl sarcosinates, acyl glutamates, or combinations thereof. Examples of amphoteric surfactants include, but are not limited to, betaines, amidopropylbetaines, or combinations thereof. Other contemplated components include the long chain amido amines, such as stearamidopropyl dimethylamine (SAPDMA). Surfactant amounts in the product composition can range from about 0.01% to about 20% by weight of the product composition.

Examples of additives include rheological modifiers, emollients, skin conditioning agents, sun care additives, emulsifier/suspending agents, thickeners, fragrances, colors, pigments, opacifiers, insect repellant actives, herbal extracts, vitamins, builders, enzymes, preservatives, antibacterial agents, pH adjusters, or combinations thereof. Particular examples of such additives include, but are not limited to, linear or branched, saturated or unsaturated alcohols having between 8 and 22 carbon atoms, silicones, siloxanes, mineral oils, natural or synthetic waxes, polyglycerol alkyl esters, glycol esters, esters of fatty acids with alcohols of low carbon number, for example isopropanol, benzoic acid esters, citric acid, succinic acid, phosphoric acid, sodium hydroxide, sodium carbonate, vitamins, such as Vitamin A, Vitamin E, or pantothenic acid, quaternized guar, celluloses or quaternized celluloses, or combinations of any of the foregoing. Total additives in the product composition can range from about 0.01% to about 40% by weight of the product composition.

Compositions of the present technology, comprising the dialkyl amino acid ester salt, provide several benefits. The dialkyl amino acid ester salts have a BCI of at least 80, alternatively at least 90, and preferably of 100, meaning they can be derived entirely from natural sources. Having a BCI of 100 provides a benefit from an environmental standpoint, since such components are more environmentally friendly than components derived from petroleum sources. The hair conditioning formulations comprising the dialkyl amino acid ester salts provide better wet hair combing properties compared to formulations comprising Brassicyl L-Isoleucine esylate, a known neutralized amino acid ester, at comparable use levels. In addition, combining a glyceride component with the dialkyl amino acid ester salts of the present technology can further improve the wet combing properties of a hair care composition. Glycerides can be derived entirely from biorenewable sources, and therefore can have a BCI of 100. Thus, the combination of dialkyl amino acid ester salt and glyceride can also have a BCI of 100, and can deliver improved performance with an environmentally friendly profile. In addition, since the glyceride component is a less costly ingredient than the dialkyl amino acid ester salt component, the mixture of the dialkyl amino acid ester salt and glyceride component can provide better performance properties at a lower cost, compared to formulations comprising the dialkyl amino acid ester salt alone, and also compared to formulations comprising Brassicyl L-Isoleucine esylate. The hair conditioning formulations also provide better wet hair combing properties compared to formulations comprising CETAC. However, unlike CETAC, the dialkyl amino acid ester salts of the present technology are biodegradable, and provide an improved environmental profile and lower toxicity compared to CETAC. The improved performance compared to CETAC is surprising, because typical cationic conditioning agents contain fatty carbon chains that are primarily C16/C18 carbon atoms or greater, whereas the dialkyl amino acid ester salts of the present technology have fatty carbon chains that are primarily C12/C14 carbon atoms.

Hair conditioning compositions comprising the alkyl amino acid ester salt component of the present technology can be applied to the hair in an amount suitable for obtaining a hair conditioning effect. Suitable amounts of the dialkyl amino acid ester salt as a conditioning active applied to the hair can range from about 0.001% to about 5% by weight, alternatively about 0.001% to about 2%, alternatively about 0.002% to about 1.5%, alternatively about 0.025% to about 0.5%, alternatively about 0.025% to about 0.25% by weight, as measured on dry hair. The hair conditioning compositions provide a wet combing Dia-Stron Maximum Peak Load of about 55 gram mass force (gmf) or less, alternatively about 50 gmf or less, alternatively about 45 gmf or less, such as about 20 to about 40 gmf. The hair conditioning formulas of the present invention have a pH of between 3 and 6, alternatively between 3.2 and 5.2, alternatively between 3.5 and 4.5.

### EXAMPLES

The presently described technology and its advantages will be better understood by reference to the following examples. These examples are provided to describe specific embodiments of the present technology. By providing these examples, the inventors do not limit the scope and spirit of the present technology.

The following test methods are used to determine properties and performance of compositions of the present technology.

### Dia-Stron Procedure for Wet and Dry Combing

1. Rinse tress for 30 seconds.

2. Apply 0.5 mL of VO5® Volumizing Shampoo (non-conditioning shampoo).

3. Spread throughout tress.

4. Allow to air-dry.

5. Rinse tress for 30 seconds.

6. Apply 0.5 mL of Test Conditioner.

7. Spread throughout tress.

8. Rinse tress for 30 seconds.

9. Affix tress to Dia-Stron MT1775 instrument and run “Wet Combing” procedure.

10. Repeat Step 9 nine more times for one tress.

11. Repeat Step 1-10 for 2 more tresses.

12. Allow tresses to air-dry.

13. Affix tress to Dia-Stron MT1775 instrument and run “Dry Combing” procedure.

14. Repeat Step 13 nine more times for one tress.

15. Repeat Steps 13-14 for 2 more tresses.

### Example 1: Synthesis of Dilaurylaspartate Ethanyl Sulfonate (2:1 Ratio)

Molten lauryl alcohol (281.04 g, 2 equiv.), and L-aspartic acid (99.90 g, 1 equiv.) were charged to a 1 L, 4-necked reaction flask equipped with a mechanical stirrer, a thermocouple, and a short-path distillation head attached to a mineral oil filled bubbler. A tared 100 mL round-bottomed flask was installed on the distillation head as a receiver. A 250 mL pressure-equalized addition funnel containing 129.02 g (1.1 equiv.) of 70% ethanesulfonic acid aqueous solution was attached to the remaining neck of the flask and a nitrogen source was attached atop the addition funnel. The system was placed under a nitrogen headspace sweep and heated to 40° C. to ensure that the lauryl alcohol remained molten. The acid was added dropwise over a period of 65 minutes. Once the addition was complete, the reaction mixture was allowed to stir at 40° C. for 20 minutes and then gradually heated to 120° C. Once at 120° C., the mixture became almost homogeneous with small amounts of a solid in the bottom of the flask. Distillation of a condensate occurred with a head temperature of 97° C. The reaction mixture was allowed to stir at 120° C. for 2.5 hours then 140° C. for a total of 13 hours. At this point, ¹H NMR indicates 86% conversion of the alcohol. The reaction was cooled to 80° C., and the reaction mixture was poured into a tared Pyrex baking dish and left in a fume hood to solidify. The material did not solidify once it reached room temperature, so the material was transferred from the dish to a tared 32 oz. wide mouth glass sample jar. The material begins to solidify after standing. A total of 421.69 g of product was isolated. This product is labelled LAES2:1

### Example 2: Synthesis of Dilaurylaspartate Ethanyl Sulfonate (3:1 Ratio)

Molten lauryl alcohol (428.04 g, 3 equiv.), and L-aspartic acid (99.87 g, 1 equiv.) were charged to a 1 L, 4-necked reaction flask equipped with a mechanical stirrer, a thermocouple, and a short-path distillation head attached to a mineral oil filled bubbler. A tared 300 mL round-bottomed flask was installed on the distillation head as a receiver. A 250 mL pressure-equalized addition funnel containing 129.04 g (1.1 equiv.) of 70% ethanesulfonic acid aqueous solution was attached to the remaining neck of the flask and a nitrogen source was attached atop the addition funnel. The system was placed under a nitrogen headspace sweep and heated to 40° C. to ensure that the lauryl alcohol remained molten. Once at 40° C., the acid solution was added dropwise over a period of 1.5 hours. No noticeable exotherm was observed. Once the addition was complete, the reaction mixture was allowed to stir at 40° C. for 15 minutes and then gradually heated to 120° C. in 10-20° C. increments. After a few minutes at 120° C., the mixture became homogeneous. Distillation of a condensate occurred with a head temperature of 97° C. and solidified in the condenser requiring the cooling water be turned off to allow it to melt and collect in the receiver. The reaction mixture was allowed to stir at 120° C. for 2.5 hours then heated to 140° C. under a 100 mL/min flow of nitrogen, collecting any distillate in the same receiver, and held in this state for 7 hours. ¹H NMR indicates that the conversion of alcohol is 65.5%, or roughly ⅔ of the original charge. The reaction product was transferred at 80° C. to a tared, labeled, 1 quart glass jar. A total of 580.74 g of material was transferred to the jar. This product is designated LAES3:1

### Example 3: Synthesis of Dilaurylaspartate Methanyl Sulfonate (2:1 Ratio)

Molten lauryl alcohol (286.33 g, 2 equiv.), and L-aspartic acid (100.62 g, 1 equiv.) were charged to a 1 L, 4-necked reaction flask equipped with a mechanical stirrer, a thermocouple, and a short-path distillation head attached to a mineral oil filled bubbler. A tared 100 mL round-bottomed flask was installed on the distillation head as a receiver. A 60 mL pressure-equalized addition funnel containing 79.55 g (1.1 equiv.) of methanesulfonic acid was attached to the remaining neck of the flask and a nitrogen source was attached atop the addition funnel. The system was placed under a nitrogen headspace sweep, and the acid was added dropwise over a period of 1.5 hours. The temperature of the reaction mixture started at 42° C. (due to the hot lauryl alcohol) and remained between 45-46° C. during the addition of the acid. Once the addition was complete, the reaction mixture was heated to 140° C. Once at 140° C., the mixture became almost homogeneous with small amounts of a solid in the bottom of the flask. The reaction mixture was allowed to stir at 140° C. for a total of 15 hours after which the conversion of alcohol in the reaction determined by ¹H NMR is 92.8% yielding 427.7 g of product that solidifies upon standing.

This product is designated LAMS. While the synthetic route using methanesulfonic acid is not preferred due to agglomeration of the reaction mixture, the end molecule is a preferred option.

### Example 4: Synthesis of Lauryl/Myristyl Aspartate Ethanyl Sulfonate (2:1 Ratio)

Molten fatty alcohol, CepSinol® 1216 (292.68 g, 2 equiv, OHV = 288 mg KOH/g, EW = 194.79, with approximate carbon chain distribution of 70% C12, 25% C14 and 5% C16), and L-aspartic acid (99.98 g, 1 equiv.) were charged to a 1 L, 4-necked reaction flask equipped with a mechanical stirrer, a thermocouple, and a short-path distillation head attached to a mineral oil filled bubbler. A tared 300 mL round-bottomed flask was installed on the distillation head as a receiver. A 250 mL pressure-equalized addition funnel containing 129 g (1.1 equiv.) of 70% ethanesulfonic acid aqueous solution was attached to the remaining neck of the flask and a nitrogen source was attached atop the addition funnel. The system was placed under a 100 mL/min nitrogen headspace sweep and heated to 40° C. to ensure that the alcohol remained molten. The acid was added dropwise over a period of 50 minutes. The reaction was gradually heated to 120° C. in 10-20° C. increments, holding at each temperature for 5 to 10 minutes before increasing. Once at 120° C., distillation began with a head temperature of 97° C. After approximately 30 minutes, the head temperature decreased, and the distillation stopped. The reaction temperature was increased to 140° C. and held for 1.5 hours. At this point, a total of 44.11 g of condensate (80.2% of the theoretical 54.99 g (theoretical value includes water in the acid solution)) had been collected. After an additional 1 hour at 140° C., no further distillation occurred, so the nitrogen headspace sweep was converted to a nitrogen sparge at 200 mL/min and the reaction mixture was heated at 140° C. for an additional 12.5 hours. At this point, ¹H NMR indicates that the conversion of alcohol is 90.5%. The reaction mixture is cooled to 80° C. and transferred to a sample jar. A total of 448.80 g of product that slowly solidifies upon standing at room temperature is obtained.

This product is designated LMAES2:1

### Example 5: Synthesis of Lauryl/Myristyl Aspartate Ethanyl Sulfonate (3:1 Ratio)

Molten fatty alcohol, CepSinol® 1216 (439.05 g, 3 equiv, OHV = 288 mg KOH/g, EW = 194.79, with approximate carbon chain distribution of 70% C12, 25% C14 and 5% C16), and L-aspartic acid (100.02 g, 1 equiv.) were charged to a 1 L, 4-necked reaction flask equipped with a mechanical stirrer, a thermocouple, and a short-path distillation head attached to a mineral oil filled bubbler. A tared 300 mL round-bottomed flask was installed on the distillation head as a receiver. A 250 mL pressure-equalized addition funnel containing 129.08 g (1.1 equiv.) of 70% ethanesulfonic acid aqueous solution was attached to the remaining neck of the flask and a nitrogen source was attached atop the addition funnel. The system was placed under a 100 mL/min nitrogen headspace sweep and heated to 40° C. to ensure that the alcohol remained molten. The acid was added dropwise over a period of 50 minutes, and the mixture was held at 40° C. for 30 minutes. The reaction was heated to 120° C., and once at 120° C., distillation began with a head temperature of 93° C. After approximately 45 minutes, the head temperature decreased, and the distillation stopped. The reaction temperature was increased to 140° C. and held for 3.75 hours under a 250 mL/min headspace purge of nitrogen. At this point, a total of 58.33 g of condensate (89.7% of the theoretical 54.99 g (theoretical value includes water in the acid solution)) had been collected. After an additional 7 hours at 140° C., ¹H NMR indicates that the conversion of alcohol is 72%. The reaction mixture is transferred to a sample jar while at 60° C. A total of 598.57 g of product that slowly solidifies upon standing at room temperature is obtained.

This product is designated LMAES3:1

### Example 6: Synthesis of Dilaurylglutamate Ethanyl Sulfonate (2:1 Ratio)

Molten lauryl alcohol (250.0 g, 2 equiv.) and L-glutamic acid (100.0 g, 1 equiv.) were charged to a 1 L, 4-necked reaction flask equipped with a mechanical stirrer, thermocouple, and 250 mL pressure-equalizing addition funnel containing 116.6 g (1.1 equiv.) of a 70.6% ethanesulfonic acid aqueous solution. The system was placed under a nitrogen headspace sweep and heated to 45° C. to ensure that the lauryl alcohol remained molten. The acid was added dropwise over a period of 35 minutes. Once the addition was complete, the pressure-equalizing dropping funnel was exchanged for a short-path distillation head connected to a mineral oil bubbler and fitted with a tared 100 mL round bottom flask. The reaction mixture was then gradually heated to 140° C. Once at 120° C., the mixture became almost homogeneous with small amounts of a solid in the bottom of the flask. Distillation of a condensate occurred with a head temperature of 97° C. and a total of 40.6 g of distillate containing oil droplets was collected. The reaction mixture was allowed to stir at 140° C. for a total of 20 hours. The reaction was cooled to 80° C. and then poured into a tared 32 oz. wide mouth glass sample jar. The material begins to solidify after standing. A total of 399.2 g of product was isolated. This product is designated LGES2:1

### Example 7: Synthesis of Dilaurylglutamate Ethanyl Sulfonate (3:1 Ratio)

Molten lauryl alcohol (376.1 g, 3 equiv.) and L-glutamic acid (100.0 g, 1 equiv.) were charged to a 1 L, 4-necked reaction flask equipped with a mechanical stirrer, thermocouple, and 250 mL pressure-equalizing addition funnel containing 116.6 g (1.1 equiv.) of a 70.6% ethanesulfonic acid aqueous solution. The system was placed under a nitrogen headspace sweep and heated to 45° C. to ensure that the lauryl alcohol remained molten. The acid was added dropwise over a period of 35 minutes. Once the addition was complete, the pressure-equalizing dropping funnel was exchanged for a short-path distillation head connected to a mineral oil bubbler and fitted with a tared 100 mL round bottom flask. The reaction mixture was then gradually heated to 140° C. Once at 120° C., the mixture became almost homogeneous with small amounts of a solid in the bottom of the flask. Distillation of a condensate occurred with a head temperature of 97° C. and a total of 44.0 g of distillate containing oil droplets was collected. The reaction mixture was allowed to stir at 140° C. for a total of 20 hours. The reaction was cooled to 80° C. and then poured into a tared 32 oz. wide mouth glass sample jar. The material begins to solidify after standing. A total of 403.7 g of product was isolated. This product is designated LGES3:1

### Example 8: Synthesis of Lauryl/Myristyl Glutamate Ethanyl Sulfonate (2:1 Ratio)

Molten fatty alcohol, CepSinol® 1216 (456.4 g, 2 equiv, OHV = 288 mg KOH/g, EW = 194.79, with approximate carbon chain distribution of 70% C12, 25% C14 and 5% C16) and L-glutamic acid (175.0 g, 1 equiv.) were charged to a 2 L, 4-necked reaction flask equipped with a mechanical stirrer, thermocouple, and 250 mL pressure-equalizing addition funnel containing 204.0 g (1.1 equiv.) of a 70.6% ethanesulfonic acid aqueous solution. The system was placed under a nitrogen headspace sweep and heated to 45° C. to ensure that the alcohol remained molten. The acid was added dropwise over a period of 35 minutes. Once the addition was complete, the pressure-equalizing dropping funnel was exchanged for a short-path distillation head connected to a mineral oil bubbler and fitted with a tared 100 mL round bottom flask. The reaction mixture was then gradually heated to 140° C. Once at 120° C., the mixture became almost homogeneous with small amounts of a solid in the bottom of the flask. Distillation of a condensate occurred with a head temperature of 97° C. and a total of 96.4 g of distillate containing oil droplets was collected. The reaction mixture was allowed to stir at 140° C. for a total of 20 hours. The reaction was cooled to 80° C. and then poured into a tared 32 oz. wide mouth glass sample jar. The material begins to solidify after standing. A total of 710.8 g of product was isolated. This product is designated LMGES2:1

### Example 9: Synthesis of Lauryl/Myristyl Glutamate Ethanyl Sulfonate (3:1 Ratio)

Molten CepSinol® 1216 (691.1 g, 3 equiv, OHV = 288 mg KOH/g, EW = 194.79, with approximate carbon chain distribution of 70% C12, 25% C14 and 5% C16),) and L-glutamic acid (175.0 g, 1 equiv.) were charged to a 2 L, 4-necked reaction flask equipped with a mechanical stirrer, thermocouple, and 250 mL pressure-equalizing addition funnel containing 204.0 g (1.1 equiv.) of a 70.6% ethanesulfonic acid aqueous solution. The system was placed under a nitrogen headspace sweep and heated to 45° C. to ensure that the alcohol remained molten. The acid was added dropwise over a period of 35 minutes. Once the addition was complete, the pressure-equalizing dropping funnel was exchanged for a short-path distillation head connected to a mineral oil bubbler and fitted with a tared 100 mL round bottom flask. The reaction mixture was then gradually heated to 140° C. Once at 120° C., the mixture became almost homogeneous with small amounts of a solid in the bottom of the flask. Distillation of a condensate occurred with a head temperature of 97° C. and a total of 88.7 g of distillate containing oil droplets was collected. The reaction mixture was allowed to stir at 140° C. for a total of 20 hours. The reaction was cooled to 80° C. and then poured into a tared 32 oz. wide mouth glass sample jar. The material begins to solidify after standing. A total of 943.0 g of product was isolated. This product is designated LMGES3:1

### Example 10: Synthesis of Stearyl/Oleyl Aspartate Methanyl Sulfonate (Comparative)

Stearyl alcohol (151.04 g, OHV 209.2 mg KOH/g, 1 equiv.), oleyl alcohol (153.52 g, OHV = 206 mg KOH/g, 1 equiv.), and L-aspartic acid (74.90 g, 1 equiv.) were charged to a 1 L, 4-necked reaction flask equipped with a mechanical stirrer, a thermocouple, and a short-path distillation head attached to a mineral oil filled bubbler. A tared 300 mL round-bottomed flask was installed on the distillation head as a receiver. A 100 mL pressure-equalized addition funnel containing 59.62 g (1.1 equiv. on aspartic acid) of methanesulfonic acid was attached to the remaining neck of the flask and a nitrogen source was attached atop the addition funnel. The system was placed under a nitrogen headspace sweep, and the contents of the reactor were heated to 90° C. Once at 90° C. and the alcohol mixture appeared to be completely molten, the methanesulfonic acid was added dropwise over 50 minutes. The mixture turned dark brown (almost black), and as the system stirred, the aspartic acid congealed and collapsed into a ball making the mixture hard to stir. The temperature was increased in 10° C. increments to a final temperature of 140° C. Once at 120° C., the “ball” of aspartic acid broke up and began to dissolve. Once at 140° C., the mixture became almost homogeneous with small amounts of a solid in the bottom of the flask. The reaction mixture was allowed to stir at 140° C. for a total of 17.25 hours after which the receiver was chilled in dry ice and the reaction was allowed to stir at 140° C. for an additional 6 hours. At this point, 1H NMR indicates 97.5% conversion of the alcohol.

The reaction mixture was allowed to cool to 75° C. (system begins to solidify below 75° C.), and 12.5 g of 25% methanolic sodium methoxide was added over 20 minutes via a pressure equalized addition funnel to quench the excess methanesulfonic acid used. Once the addition was complete, the mixture was allowed to stir at 75° C. for 45 minutes. The receiver was chilled in dry ice, and intermittent vacuum was applied to prevent overflow into the condenser head from the vigorous frothing that began to occur. Intermittent vacuum was continued until no further frothing occurred and then the system was left under full vacuum with heating to 85° C. and held for 1 hour yielding 402.26 g of a dark brown liquid product that solidifies upon cooling.

The product is very dark and resinous. While not wishing to be bound by theory, it is believed that the double bonds in the oleyl alcohol are protonated during the reaction causing migration of the double bonds along with a number of unwanted side reactions. Therefore, it is desirable to minimize the amount of double bonds in the alcohol reactant. This product is designated SOAMS and is not within the scope of the invention.

### Example 11: Synthesis of Dicocoyl Glutamate Ethanyl Sulfonate (CGES2:1 Ratio)

A mixture of alcohols was formulated to mimic the distribution of whole coconut alcohol and had the following carbon chain distribution: C8 (6.22%); C10 (5.76%); C12 (45.85%); C14 (19.60%); C16 (9.73%); C18 (12.79%); C20 (0.04%). The EW of this product is 196.08 g/mol. To a 2 L four neck flask fitted with an overhead stirrer, nitrogen inlet and thermocouple was charged “Formulated Whole coconut alcohol” (251.3 g, 1282 mmol, 2 equiv., 100 mass%) and L-glutamic acid (94.22 g, 640.4 mmol, 1 equiv., 100 mass%). This mixture was warmed under a flow of nitrogen to 45° C. and the apparatus fitted with an 250 mL pressure-equalizing addition funnel charged with 70.6% ethanesulfonic acid aqueous solution (110.65 g, 709 mmol, 70.6 mass%). The ethanesulfonic acid was added slowly over the course of 1 hr, giving rise to a colorless precipitate that is well distributed in solution. There is no exothermic event upon addition of the ethanesulfonic acid solution with the mixture. Once the addition is complete, the dropping funnel is exchanged for a short path distillation head fitted with a tarred flask to monitor water evolution. The temperature is raised in 20° C. increments over the course of 2 hours to 140° C., and held at this temperature for a total of 26 hours after which the reaction is judged complete by ¹H NMR. The reaction mixture is transferred to a sample jar yielding 379 g of a pale yellow mixture. The distillate from the short path distillation head weighs 54.4 g.

### Example 12: Synthesis of Dicocoyl Glutamate Ethanyl Sulfonate (CGES3:1 Ratio)

To a 2L four neck flask fitted with an overhead stirrer, nitrogen inlet and thermocouple was charged formulated whole coconut alcohol as described in Example 11 (287.0 g, 1464 mmol, 3 equiv., 100 mass%) and L-glutamic acid (71.78 g, 487.9 mmol, 1 equiv., 100 mass%). This mixture was warmed under a flow of nitrogen to 45° C. and the apparatus fitted with an 250 mL pressure-equalizing addition funnel charged with 70.6% ethanesulfonic acid aqueous solution (84.9 g, 544 mmol, 70.6 mass%). The ethanesulfonic acid was added slowly over the course of 1 hr, giving rise to a colorless precipitate that is well distributed in solution. There is no exothermic event upon action of the ethanesulfonic acid solution with the mixture. Once the addition is complete, the dropping funnel is exchanged for a short path distillation head fitted with a tarred flask to monitor water evolution. The temperature is raised in 20° C. increments over the course of 2 hours to 140° C. and held at this temperature for a total of 26 hours after which the reaction is judged complete by ¹H NMR. The reaction mixture is transferred to a sample jar yielding 386 g of colorless mixture. The distillate from the short path distillation head weighs 62.6 g

### Example 13: Synthesis of Dicocoyl Glutamate Ethanyl Sulfonate Without C8′s and C10′s (CGES2:1 Ratio)

A mixture of alcohols was formulated to mimic the distribution of whole coconut alcohol without C8 and C10 alcohols and had the following carbon chain distribution: C8 (0%); C10 (0.07%); C12 (52.37%); C14 (22.31%); C16 (10.60%); C18 (14.59%); C20 (0.05%). The EW of this product is 207.17 g/mol. To a 2L four neck flask fitted with an overhead stirrer, nitrogen inlet and thermocouple was charged formulated coconut alcohol (no C8/C10) (289.04 g, 1395.2 mmol, 2 equiv., 100 mass%) and L-glutamic acid (102.6 g, 697.3 mmol, 1 equiv., 100 mass%). This mixture was warmed under a flow of nitrogen to 45° C. and the apparatus fitted with an 250 mL pressure-equalizing addition funnel charged with 70.6% ethanesulfonic acid aqueous solution (120.6 g, 773 mmol, 70.6 mass%). The ethanesulfonic acid was added slowly over the course of 1 hr, giving rise to a colorless precipitate that is well distributed in solution. There is no exothermic event upon action of ethanesulfonic acid with the mixture. One the addition is complete, the dropping funnel is exchanged for a short path distillation head fitted with a tarred flask to monitor water evolution. The temperature is raised in 20° C. increments over the course of 2 hours to 140° C., and held at this temperature for a total of 30 hours after which the reaction is judged complete by ¹H NMR.

### Example 14: Synthesis of Dicocoyl Glutamate Ethanyl Sulfonate Without C8′s and C10′s (CGES3:1 Ratio)

To a 2 L four neck flask fitted with an overhead stirrer, nitrogen inlet and thermocouple was formulated whole coconut alcohol (no C8/C10) as described in Example 13 (326.9 g, 1578 mmol, 3 equiv., 100 mass%) and L-glutamic acid (77.71 g, 528.2 mmol, 1 equiv., 100 mass%). This mixture was warmed under a flow of nitrogen to 45° C. and the apparatus fitted with an 250 mL pressure-equalizing addition funnel charged with 70.6% ethanesulfonic acid aqueous solution (92.0 g, 590 mmol, 70.6 mass%). The ethanesulfonic acid was added slowly over the course of 1 hr, giving rise to a colorless precipitate that is well distributed in solution. There is no exothermic event upon action of ethanesulfonic acid with the mixture. Once the addition is complete, the dropping funnel is exchanged for a short path distillation head fitted with a tarred flask to monitor water evolution. The temperature is raised in 20° C. increments over the course of 2 hours to 140° C., and held at this temperature for a total of 24 hours after which the reaction is judged complete by ¹H NMR.

### Example 15: Synthesis of Distearyl Glutamate Ethanyl Sulfonate (SGES2:1 Ratio)

To a 1L four neck RBF fitted with overhead stirring, nitrogen inlet, and short path distillation head (vented to external bubbler) was charged with stearyl alcohol ( 362.18 g, 1339 mmol, 2 equiv., 100 mass%) and L-glutamic acid (100 g, 679.67 mmol, 1 equiv., 100 mass%) and the mixture heated to 70-75° C. to give a slurry of glutamic acid in stearyl alcohol. To this mixture was added 70.6% ethanesulfonic acid aqueous solution (117 g, 750 mmol, 70.6 mass%) by means of a pressure equalizing dropping funnel over the course of 30 min. This results in the formation of a soft white precipitate that resists agglomeration in solution. After the addition is complete, the dropping funnel is removed, and the temperature increased to 140° C. over the course of 6 hours in 10-20° C. increments. After reaching 140° C., the solid has been completely consumed in the reaction mixture and it is now homogeneous. The reaction mixture is stirred for a total of 21 hours at 140° C., after which the reaction is judged complete by ¹H NMR. The molten reaction mixture is transferred to a sample jar yielding 502.3 g of product.

In each of the above synthesis examples, excess ethanyl sulfonate or methanyl sulfonate can be neutralized with a suitable base, such as, for example, sodium carbonate. Also, for each of the reaction products prepared in the above examples, the % active is the combined total of the monoester and diester salt species present in the reaction product. The different species present in each reaction product were determined by NMR.

### Example 16: Preparation of Hair Conditioning Composition

Hair conditioning compositions were formulated in accordance with the General Procedure below, using dialkyl amino acid ester salts of the present technology, alone, as the conditioning active, or in combination with a glyceride component as the conditioning active. The glyceride component was DREWMULSE® GMO (hereinafter “GMO”), a glycerol oleate comprising mono- and diglyceryl oleates in a ratio of about 1:1, available from Stepan Company, Northfield, Illinois. Table 1 shows the general formula used to make the hair conditioning compositions.

TABLE 1

| | | |
|---|---|---|
| Material Chemical Name | Function | %W/W in Formulation |
| Deionized Water | Carrier | q.s. to 100.0’ |
| Natrosol™ 250 HHR CS (Hydroxyethylcellulose) | Thickener | 0.7 |
| Sodium Hydroxide | pH adjuster | q.s. |
| Conditioning Agent | Conditioning Active | Per examples which follow |
| Cetyl Alcohol | Viscosity Modifier | 2.0 |
| Potassium Chloride, 10% Solution | Opacifier | 0.5 |
| Citric Acid | pH adjuster | q.s. |
| Kathon™ CG | Preservative | q.s. |

### General Procedure

1. Charge water, begin mixing
2. Sprinkle in Natrosol 250 HHR CS
3. Adjust pH with 25% Sodium Hydroxide to target of pH 8-9. Mix until clear (30-40 min)
4. Heat to 70-75° C.
5. Add Conditioning component and mix until homogenous
6. Add Cetyl Alcohol and mix for 30 min.
7. Cool to 45° C. with mixing
8. In a small beaker dissolve Potassium Chloride in Water. Add to batch
9. Adjust pH 3.5-4 with 50% Citric Acid
10. Cool to Room Temp.
11. Add Kathon CG

The hair conditioning formulations used in the following examples were prepared in accordance with the Table 1 formulation and the General Procedure. Amounts in the following Tables are based on weight %. BTAC refers to behentrimonium chloride, CETAC refers to cetrimonium chloride (AMMONYX® CETAC-30 from Stepan Company, Northfield, Illinois), GMO refers to DREWMULSE® GMO, a glycerol oleate comprising mono- and diglyceryl oleates in a ratio of about 1:1, and brassicyl L-isoleucinate ethanylsulfate (“BLIE”), a neutralized amino acid ester that is the reaction product of neutralized L-isoleucine reacted with brassica alcohol and prepared according to the procedure of Example 1 of U.S. Pat. No. 8,105,569 to Burgo. Each composition (comparative and inventive) is formulated to contain 2% by weight total conditioning active - if GMO is present, it is included as part of the conditioning active. Each of the hair conditioning compositions was evaluated for wet combing ability using the Dia-Stron MTT175 instrument and the wet combing procedure.

### Example 17: Comparative Conditioning Agents

Comparative hair conditioning compositions were prepared in accordance with the Table 1 formulation and following the General Procedure, except that different cationic surfactants or amine salts were used as the only conditioning active, instead of dialkyl amino acid ester salts of the present invention. The comparative conditioning actives were BTAC, CETAC and BLIE. Each of the hair conditioning compositions was evaluated for wet combing ability using the Dia-Stron MTT175 instrument and the wet combing procedure. Results are provided in Table 2.

TABLE 2

| Conditioning Agent | % Active in conditioning agent | Amount of “as is” conditioning agent used in formula (%) | Total active in formula (%) | Maximum peak load (gmf) |
|---|---|---|---|---|
| BTAC | 70 | 2.86 | 2.0 | 19.9 |
| CETAC | 30 | 6.67 | 2.0 | 68.7 |
| BLIE | 100 | 2.0 | 2.0 | 155.8 |

As shown in Table 2, commonly used conditioning actives, namely BTAC and CETAC, yield Dia-Stron maximum peak load results of about 20 gmf and 70 gmf, respectively, when used at 2% active in a typical formula. BLIE, an amino acid-based conditioning salt, yielded a maximum peak load of about 156 gmf.

### Example 18: Inventive Conditioning Agent From Example 1

Dia-Stron wet combing results for hair conditioning formulas according to Table 1 and utilizing the Example 1 dialkyl amino acid ester salt (LAES2:1) as the conditioning agent by itself and in combination with GMO are provided in Table 3.

TABLE 3

| Conditioning Agent | % Active in conditioning agent | Amount of “as is” conditioning agent used in formula (%) | Total active in formula (%) | Maximum peak load (gmf) |
|---|---|---|---|---|
| LAES2:1 | 76.9 | 2.60 | 2.0 | 42.9 |
| 80% LAES2:1/20% GMO | 76.9/100 | 2.08 LAES2:1/0.4 GMO | 2.0 | 35.9 |

The hair conditioner containing LAES2:1 had a Dia-Stron maximum peak load of about 43 gmf, which is an improvement over the results obtained using CETAC. These results show that the dialkyl amino acid ester salt of the present technology can provide better wet combing properties than CETAC, a commonly used cationic conditioning agent (see CETAC results in Table 2). The results in Table 3 also show that a hair conditioning formula according to Table 1 containing 2% active LAES2:1 provides greatly improved wet combing properties compared to the composition comprising 2% BLIE, a known neutralized amino acid ester (see BLIE results in Table 2). The composition comprising BLIE as the conditioning agent, which has a BCI of 100, had a Dia-Stron maximum peak load of about 156 gmf, compared to the about 43 gmf maximum peak load achieved by the composition comprising the inventive dialkyl amino acid ester salt (LAES2:1) of the present technology, which also has a BCI of 100. These results demonstrate that conditioning performance does not need to be sacrificed when using a conditioning component that has a BCI of 100 (i.e. all of the carbons are derived from a biorenewable source).

The results in Table 3 further show that combining the dialkyl amino acid ester salt of the present technology with a glyceride component can improve the wet combing properties of the composition. The composition comprising the combination of LAES2:1 and glycerides had a Dia-Stron maximum peak load of about 36 gmf, compared to the about 43 gmf maximum peak load of the composition comprising LAES2:1 alone as the conditioning agent. It will also be appreciated that the glycerides are a less costly ingredient that the dialkyl amino acid ester salt. Thus, the mixture of glycerides with the dialkyl amino acid ester salt not only improves the wet combing properties of the cationic active, but also reduces the cost of the cationic active in the hair care composition.

### Example 19: Inventive Conditioning Agent From Example 2

Dia-Stron wet combing results comprising 2% by active weight of the Example 2 dialkyl amino acid ester salt (LAES3:1) as the conditioning agent by itself and in combination with GMO are provided in Table 4.

TABLE 4

| Conditioning Agent | % Active in conditioning agent | Amount of “as is” conditioning agent used in formula (%) | Total active in formula (%) | Maximum peak load (gmf) |
|---|---|---|---|---|
| LAES3:1 | 65.4 | 3.06 | 2.0 | 36.6 |
| 90% LAES3:⅒% GMO | 65.4/100 | 2.75 LAES3:1/0.2 GMO | 2.0 | 33.0 |

The composition comprising LAES3:1 as the conditioning agent had a Dia-Stron maximum peak load of about 36 gmf, and composition comprising the combination of LAES3:1 and glycerides had a Dia-Stron maximum peak load of about 33 gmf. The results in Table 4 show that the composition comprising LAES3:1 as the conditioning agent provides better wet combing results than either of the compositions containing CETAC or BLIE as the conditioning agent. (Compare Table 4 with Table 2). Combining glycerides with LAES3:1 provides a slight improvement in the wet combing properties of the composition compared to LAES3:1 alone.

### Example 20: Inventive Conditioning Agent From Example 3

Dia-Stron wet combing results for hair conditioning formulas according to Table 1 and utilizing the Example 3 dialkyl amino acid ester salt (LAMS) as the conditioning agent by itself, and in combination with GMO are provided in Table 5. Since the LAMS conditioning agent comprises 85% by weight LAMS active, the formula containing 2% by weight of the LAMS conditioning agent comprises 1.7% by weight of the LAMS active, and the formula containing 2% by weight of the combination of LAMS and GMO comprises a total conditioning active amount of 1.8% by weight.

TABLE 5

| Conditioning Agent | % Active in conditioning agent | Amount of “as is” conditioning agent used in formula (%) | Total active in formula (%) | Maximum peak load (gmf) |
|---|---|---|---|---|
| LAMS | 85 | 2.0 | 1.7 | 51.9 |
| 70% LAMS/30% GMO | 85/100 | 1.4 LAMS/0.6 GMO | 1.8 | 33.0 |

The hair conditioner containing LAMS had a Dia-Stron maximum peak load of about 52 gmf, which is an improvement over the results obtained using CETAC. These results show that LAMS can provide better wet combing properties than CETAC, a commonly used cationic conditioning agent. The results in Table 5 also show that a hair conditioning formula according to Table 1 containing 1.7% by active weight dialkyl amino acid ester salt from Example 3 (LAMS) provides greatly improved wet combing properties compared to the composition comprising 2% BLIE, a known neutralized amino acid ester. The composition comprising BLIE as the conditioning agent, which has a BCI of 100, had a Dia-Stron maximum peak load of about 156 gmf, compared to the about 52 gmf maximum peak load achieved by the composition comprising the dialkyl amino acid ester salt of the present technology, which also has a BCI of 100.

The results in Table 5 further show that LAMS combined with a glyceride component can improve the wet combing properties of the composition. The composition comprising the combination LAMS and glycerides had a Dia-Stron maximum peak load of 33 gmf, compared to the about 52 gmf maximum peak load of the composition comprising LAMS alone as the conditioning agent. It will also be appreciated that the glycerides are a less costly ingredient that the dialkyl amino acid ester salt. Thus, the mixture of glycerides with the dialkyl amino acid ester salt not only improves the wet combing properties of the cationic active, but also reduces the cost of the cationic active in the hair care composition.

### Example 21: Inventive Conditioning Agent From Example 7

A hair conditioning composition was prepared according to the Table 1 formula, using 2% by weight of the Example 7 dialkyl amino acid ester salt (LGES3:1) (75.1% active) as the conditioning agent. The hair conditioning composition was evaluated for wet combing properties. The Dia-Stron wet combing result of this conditioner formula was 52.9 gmf, which is better than either the composition comprising CETAC or the composition comprising BLIE as the conditioning agent (see Table 2).

### Example 22: Conditioning Agent From Example 10 (Comparative)

SOAMS from Example 10 could not be successfully formulated into the Table 1 hair conditioner formula. Without wishing to be bound by theory, it is believed that the double bonds present from the oleyl alcohol caused excessive side reactions under the acidic reaction conditions leading to the resinous, possibly polymerized product that would not formulate well. SOAMS is therefore outside the scope of this invention. It is therefore preferred that most of the carbon chains in the alcohol used to make the dialkyl amino acid salt be saturated.

### Example 23: Inventive Conditioning Agent From Example 11

A hair conditioning composition was prepared according to the Table 1 formula, using 1.8% by weight of the Example 11 dicocoyl amino acid ester salt (CGES2:1) (75.72% active) as the conditioning agent with 0.2% by weight of DREWMULSE GMO (mono/di-glycerides) in the mixture. The hair conditioning composition was evaluated for wet combing properties. The Dia-Stron wet combing result of this conditioner formula was 42.04 gmf, which is better than either the composition comprising CETAC or the composition comprising BLIE as the conditioning agent (see Table 2).

### Example 24: Inventive Conditioning Agent From Example 12

A hair conditioning composition was prepared according to the Table 1 formula, using 1.8% by weight of the Example 12 dicocoyl amino acid ester salt (CGES3:1) (66.43% active) as the conditioning agent and 0.2% by weight of DREWMULSE GMO (mono/di-glycerides) in the mixture. The hair conditioning composition was evaluated for wet combing properties. The Dia-Stron wet combing result of this conditioner formula was 33.33 gmf, which is better than either the composition comprising CETAC or the composition comprising BLIE as the conditioning agent (see Table 2).

### Example 25: Inventive Conditioning Agent From Example 13

A hair conditioning composition was prepared according to the Table 1 formula, using 1.6% by weight of the Example 13 dicocoyl glutamate ethanyl sulfonate without C8′s and C10′s (CGES2:1 ratio) as the conditioning agent and 0.4% by weight of DREWMULSE GMO (mono/di glycerides) in the mixture. The hair conditioning composition was evaluated for wet combing properties. The Dia-Stron wet combing result of this conditioner formula was 24.48 gmf, which is better than either the composition comprising CETAC or the composition comprising BLIE as the conditioning agent (see Table 2).

### Example 26: Inventive Conditioning Agent From Example 14

A hair conditioning composition was prepared according to the Table 1 formula, using 1.6% by weight of the Example 14 dicocoyl glutamate ethanyl sulfonate without C8′s and C10′s (CGES3:1 ratio) as the conditioning agent and 0.4% by weight of DREWMULSE GMO (mono/di-glycerides) in the mixture. The hair conditioning composition was evaluated for wet combing properties. The Dia-Stron wet combing result of this conditioner formula was 24.24 gmf, which is better than either the composition comprising CETAC or the composition comprising BLIE as the conditioning agent (see Table 2).

### Example 27: Conditioning Agent From Example 15 (Comparative)

A hair conditioning composition was prepared according to the Table 1 formula, using 2.0% by weight of the Example 15 distearyl glutamate ethanyl sulfonate (SGES2:1 ratio) as the conditioning agent. The hair conditioning composition was evaluated for wet combing properties. The Dia-Stron wet combing result of this conditioner formula was 699.35 gmf, which is far worse than either the composition comprising CETAC or the composition comprising BLIE as the conditioning agent (see Table 2).

The present technology is now described in such full, clear and concise terms as to enable a person skilled in the art to which it pertains, to practice the same. It is to be understood that the foregoing describes preferred embodiments of the present technology and that modifications may be made therein without departing from the spirit or scope of the present technology as set forth in the appended claims. Further, the examples are provided to not be exhaustive but illustrative of several embodiments that fall within the scope of the claims.

1. A composition comprising:
(a) 0.01% to about 50% by weight of a cationic active component comprising a dialkyl amino acid ester salt having the following chemical formula: 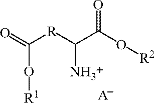
wherein R is a linear or branched carbon chain containing 1 to 10 carbon atoms, and R1 and R2 are independently C8 to C22 linear or branched alkyl groups, and A- is an anion of a proton-donating acid;
(b) optionally, one or more additional components; and
(c) diluent to balance the formulation to 100%.

2. The composition of claim 1, wherein the composition is a hair conditioning composition and comprises:
(a) 0.01% to about 50% by weight of the cationic active component;
(b) 0.01% to about 40% of one or more linear or branched alcohols having between 14 and 22 carbon atoms;
(c) optionally, one or more additional components; and
(d) diluent to balance the formulation to 100%; wherein the composition has a pH between 3 and 6.

3. The composition of claim 1, wherein the proton-donating acid is selected from the group consisting of lactic acid, citric acid, maleic acid, adipic acid, boric acid, glycolic acid, formic acid, acetic acid, ascorbic acid, uric acid, oxalic acid, butyric acid, oxalic acid, formic acid, methane sulfonic acid, ethane sulfonic acid, higher alkyl analogs of ethane sulfonic acid, hydrochloric acid, sulfuric acid, nitric acid, phosphoric acid, and combinations thereof.

4. The composition of claim 1, wherein the R1 and R2 groups are derived from an alcohol source having an iodine value of less than 3.

5. The composition of claim 1, wherein the dialkyl amino acid ester salt is dialkylaspartate ethanyl sulfonate or dialkylglutamate ethanyl sulfonate, wherein the combined R1 and R2 alkyl groups are between 65 wt% and 75 wt% C12, between 20 wt% and 30 wt% C14, and between 3 wt% and 8 wt% C16.

6. The composition of claim 1, wherein the one or more additional components comprises a glyceride component.

7. The composition of claim 6, wherein the glyceride component comprises a combination of monoglycerides and diglycerides having a carbon chain length of 8-32 carbon atoms and wherein at least 50% of the carbon chains in the monoglycerides and diglycerides in the glyceride component have at least one double bond.

8. The composition of claim 6, wherein the glyceride component and the dialkyl amino acid ester salt together have a Biorenewable Carbon Index (BCI) of at least 90.

9. The composition of claim 1, wherein the diluent comprises water, a solvent, or a combination thereof.

10. The composition of claim 2, wherein the composition, when applied to a hair tress, provides a wet combing Dia-Stron maximum peak load of about 55 gram mass force (gmf) or less.

11. The composition of claim 1, wherein the dialkyl amino acid ester salt is present in the composition in an amount of 0.01% to about 12% by weight of the composition.

12. The composition of claim 6, wherein the dialkyl amino acid ester salt and glyceride component together comprise about 0.1% to about 17% by weight of the composition.

13. The composition of claim 1, wherein the cationic active component comprises the dialkyl amino acid ester salt and a solvent.

14. The composition of claim 13, wherein the solvent is selected from the group consisting of propylene glycol, 1,3-propandiol, glycol ethers, glycerin, sorbitan esters, lactic acid, alkyl lactyl lactates, isopropyl alcohol, ethyl alcohol, dimethyl adipate, oleyl alcohol, 1,2-isopropylidine glycerol, benzyl alcohol, dimethyl lauramide myristamide, N-butyl lactate, citrate esters, dimethyl lactide, laureth-2 lactide, 1,2-butylene carbonate, conjugated linoleic acid, isosorbide dimethyl ether, propylene carbonate, C6-C18 methyl esters, C12-15 alkyl benzoate, glycerol monooleate, triglyceride oils, jojoba oil, sunflower oil/MDEA esteramine, and combinations thereof.

15. The composition of claim 14, wherein the dialkyl amino acid ester salt comprises from about 30% to about 99% by weight of the cationic active component, and the solvent comprises from about 1% to about 70% by weight of the cationic active component.

16. A method of making a dialkyl amino acid ester salt comprising the steps of:
(a) providing an amino acid having at least two carboxylic acid groups;
(b) providing a fatty alcohol feedstock, wherein the fatty alcohol feedstock comprises one or more linear or branched, saturated or unsaturated fatty alcohols having from 8 to about 22 carbon atoms;
(c) providing a proton-donating acid to protonate the amine group of the amino acid; and
(d) in the absence of added solvent, reacting the protonated amino acid with the fatty alcohol feedstock to form the dialkyl amino acid ester salt.

17. The method of claim 16, wherein the proton-donating acid is selected from the group consisting of lactic acid, citric acid, maleic acid, adipic acid, boric acid, glycolic acid, formic acid, acetic acid, ascorbic acid, uric acid, oxalic acid, butyric acid, oxalic acid, formic acid, methane sulfonic acid, ethane sulfonic acid, higher alkyl analogs of ethane sulfonic acid, hydrochloric acid, sulfuric acid, nitric acid, phosphoric acid, and combinations thereof.

18. The method of claim 16, wherein the amino acid is aspartic acid or glutamic acid.

19. The method of claim 16, wherein the fatty alcohol feedstock and the amino acid having at least two carboxylic acid groups are provided in a molar ratio of fatty alcohol to amino acid in the range of 1.6:1 to 4.5:1.

20. A dialkyl amino acid ester salt having the following chemical formula: 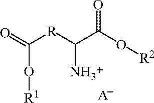
wherein R is a carbon chain containing 1 or 2 carbon atoms, R¹ and R² are independently C8 to C22 linear or branched alkyl groups, and A⁻ is ethane sulfonate or methane sulfonate.
